(19) United States
(12) Patent Application Publication
(10) Pub. No.: US 2012/0302943 A1
(43) Pub. Date: Nov. 29, 2012

(54) Systems and Methods For Deoxygenating Opthamalic Fluid

(75) Inventors: Matthew Laconte, Wildwood, MO (US); James C. Easley, Cottleville, MO (US)

(73) Assignee: SYNERGETICS, INC., O'Fallon, MO (US)

(21) Appl. No.: 13/118,061

(22) Filed: May 27, 2011

(51) Int. Cl. A61F 9/00; A61M 5/168

(52) U.S. Cl. 604/24

(57) ABSTRACT

Systems and methods for deoxygenating ophthalmic fluid are disclosed. The systems include an infusion system having a valve having an inlet in fluid communication with a source of gas, a first outlet in fluid communication with a container of ophthalmic fluid, and a second outlet in fluid communication with an infusion line of the system. The valve directs gas into the fluid disposed in the container to deoxygenate the fluid, and the deoxygenated fluid is then injected into the eye.

### FIELD

This invention generally relates to ophthalmic fluid infusion systems and, more specifically, to deoxygenating or reducing oxygen content in ophthalmic fluids introduced via an ophthalmic fluid infusion system.

### BACKGROUND

Ophthalmic fluid infusion systems may be used in a variety of ophthalmic procedures. As one example, vitrectomy is a procedure in which the vitreous within a patient's eye is removed and replaced with an ophthalmic infusion fluid, such as saline solution. An ophthalmic fluid infusion system is then used to inject the ophthalmic infusion fluid into the eye. The infusion system typically includes tubing that conveys ophthalmic infusion fluid from its container and into the eye.

The oxygen content of the ophthalmic fluid is typically greater than that of the vitreous, so it may be desirable to deoxygenate the fluid. Previous attempts to deoxygenate the fluid (i.e., reduce or eliminate the amount of oxygen in the fluid) and convey the deoxygenated fluid to the eye have not been completely satisfactory.

This Background section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### BRIEF SUMMARY

A first aspect of the present disclosure is a method of deoxygenating ophthalmic fluid injected into an eye. The method includes use of an infusion system including a valve having an inlet in fluid communication with a source of gas, a first outlet in fluid communication with a container of ophthalmic fluid, and a second outlet in fluid communication with an infusion line of the system. The method comprises actuating the valve to direct a flow of gas from the source of gas and into the fluid disposed in the container. The flow of gas deoxygenates the ophthalmic fluid. The valve is closed to stop the flow of gas into the fluid disposed in the container when the fluid has been deoxygenated below a predetermined level. The valve is then actuated to direct the flow of gas from the source of gas to the second outlet and thereby into the infusion line.

Another aspect is a method of delivering deoxygenated ophthalmic fluid into an eye. The method includes use of an infusion system including a valve having an inlet in fluid communication with a source of gas, a first outlet in fluid communication with a container of ophthalmic fluid, and a second outlet in fluid communication with an infusion line of the system. The method comprises actuating the valve to direct a flow of gas from the source of gas and into the fluid disposed in the container. The flow of gas deoxygenates the ophthalmic fluid. The valve is actuated to direct the flow of gas from the source of gas and into the infusion line when the fluid has been deoxygenated below a predetermined level. An outlet valve of the infusion line is then opened to direct a flow of deoxygenated ophthalmic fluid from the container through the infusion line and into the eye.

Still another aspect is a system for deoxygenating an ophthalmic fluid disposed in a container. The system comprises an ophthalmic infusion apparatus and a valve. The ophthalmic infusion apparatus includes a multi-lumen infusion line having a fluid outlet for discharging ophthalmic fluid into an eye and the infusion line has an inner lumen and an outer lumen. The valve has an inlet in fluid communication with a source of gas, a first outlet, and a second outlet. The valve is operable to selectively direct gas to one of the first outlet and the second outlet. The first outlet of the valve is in fluid communication with the container of ophthalmic fluid. The second outlet of the valve is in fluid communication with the infusion line adjacent the fluid outlet.

Yet another aspect is an ophthalmic fluid infusion apparatus for deoxygenating ophthalmic fluid injected into an eye. The apparatus comprises a multi-lumen infusion line, a valve, and a container of ophthalmic fluid. The infusion line has a fluid outlet for discharging fluid into the eye and an inner lumen and an outer lumen. The valve has an inlet in fluid communication with a source of gas, a first outlet, and a second outlet. The valve is operable to selectively direct gas from the inlet to one of the first outlet and the second outlet. The first outlet of the valve is in fluid communication with the container of ophthalmic fluid. gas directed from the first outlet into the fluid results in the deoxygenation of the fluid. The second outlet of the valve is in fluid communication with the ophthalmic infusion apparatus adjacent the fluid outlet of the infusion line.

Various refinements exist of the features noted in relation to the above-mentioned aspects. Further features may also be incorporated in the above-mentioned aspects as well. These refinements and additional features may exist individually or in any combination. For instance, various features discussed below in relation to any of the illustrated embodiments may be incorporated into any of the above-described aspects, alone or in any combination.

### BRIEF DESCRIPTION OF THE DRAWINGS

FIG. 1 is a perspective of an exemplary ophthalmic infusion system;

FIG. 2 is a cross-sectional view of the multi-lumen tube of FIG. 1 taken along the line 2-2;

FIG. 3 is an enlarged portion of FIG. 1;

FIG. 4 is an enlarged portion of FIG. 1;

FIG. 5 is a cross-sectional view of FIG. 4 taken along the line 5-5;

FIG. 6 is an enlarged portion of FIG. 1;

FIG. 7 is a cross-sectional view of FIG. 6 taken along the line 7-7;

FIG. 8 is a flow diagram depicting a method of deoxygenating ophthalmic fluid; and

FIG. 9 is a flow diagram depicting another method of deoxygenating ophthalmic fluid.

Like reference symbols in the various drawings indicate like elements.

### DETAILED DESCRIPTION

The embodiments described herein generally relate to systems and methods for deoxygenating ophthalmic fluid and conveying the deoxygenated fluid to the eye. Such systems will typically be used in an operating room for ophthalmic procedures such as vitrectomy.

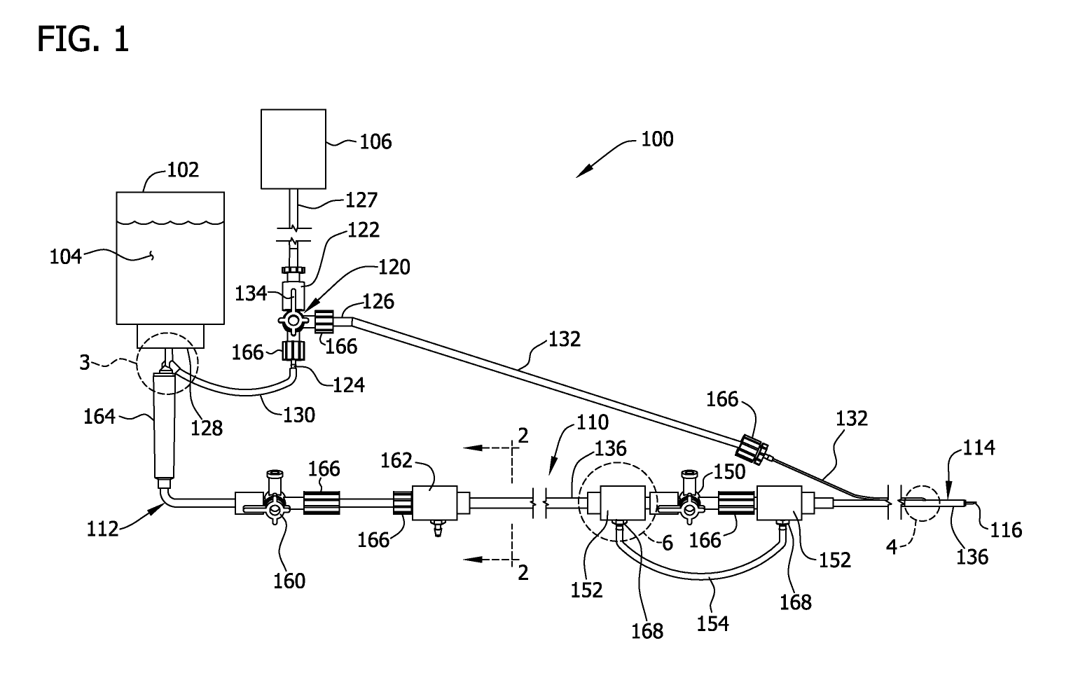

An example ophthalmic infusion system for conveying the fluid is generally indicated by reference numeral 100 in FIG. 1. The system 100 of this embodiment includes a container 102 that stores a volume of ophthalmic fluid 104. The ophthalmic fluid 104 is any suitable fluid used in ophthalmic procedures, such as saline solution, and may also be referred to generally as an intraocular irrigating solution. The system 100 may be supplied to the operating room with the container 102, or may be separately supplied and joined to the container prior to the procedure.

The system 100 also includes a source of gas 106. The source of gas 106 may be a conventional storage receptacle disposed remote from the system 100 and accessible through a wall outlet, as is customary in an operating room. In the exemplary embodiment, the gas is nitrogen, while in other embodiments the gas is any non-reactive gas (e.g., argon or helium). The relative positions of the container 102 and the source of gas 106 shown in FIG. 1 are exemplary in nature and may be changed without departing from the scope of the embodiments.

The infusion system 100 also includes an infusion line 110 generally having an inlet end 112 and an outlet end 114 opposite the inlet end. An infusion cannula 116 is connected to the infusion line 110 at the outlet end 114 in this embodiment, though other structures for injecting the fluid are contemplated within the scope of this disclosure. The infusion cannula 116 is configured to extend into a patient's eye and deliver infusion fluid 104 directly into the eye.

A three-way valve 120 is in fluid communication with the outlet end 114 of the infusion line 110, the container of ophthalmic fluid 102, and the source of gas 106. As used herein, the term “fluid communication” refers to the connection of elements by flexible tubing or other structures so that fluids such as gas and/or liquids can readily pass from one element to another element.

The three-way valve 120 (referred to interchangeably as the “valve”) has an inlet 122, a first outlet 124, and a second outlet 126. The inlet 122 is in fluid communication with the source of gas 106 via tubing 127. The inlet 122 of the valve 120 may in some embodiments also be in fluid communication with a regulator (not shown) or other similar pressure reduction device that is in turn connected to the source of gas 106.

The first outlet 124 of the valve 120 is in fluid communication with the container of ophthalmic fluid 102. A hub 128 is attached to the container of ophthalmic fluid 102 and tubing 130 extends from the hub to the first outlet 124 of the valve 120. The infusion line 110 of the infusion system 100 is also connected to the hub 128 such that the infusion line is in fluid communication with the container of ophthalmic fluid 102.

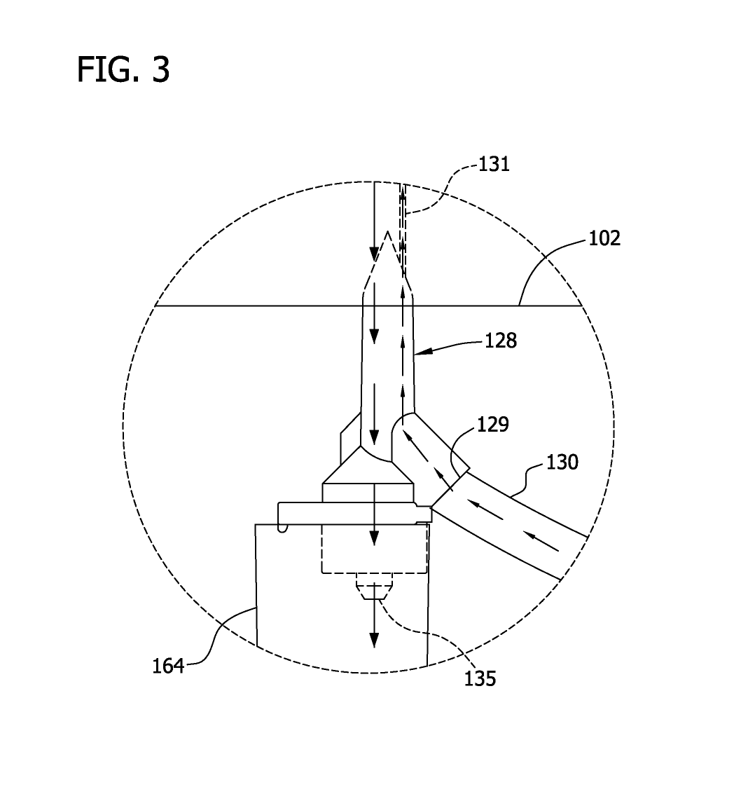

The hub 128, as best shown in FIG. 3, has an inlet 129 configured to permit gas to enter the hub. A tube 131 is connected to the inlet 129 and allows the gas to flow into the fluid 104 in the container 102. The hub 128 also has an outlet 135 to permit gas to flow out of the container 102 after it has passed through the fluid 104. The outlet also permits fluid 104 to flow out of the container 102 into a drip chamber 164. The flow of gas through the hub 128 and the inlet 129 and into the tube 131 is indicated by the left-most arrow line. The flow of gas/fluid through the hub 128 and the outlet 135 is indicated by the right-most arrow line. According to some embodiments, the container 102 and hub 128 may be inverted from the configuration shown in FIG. 1 while deoxygenating the fluid 104 contained therein.

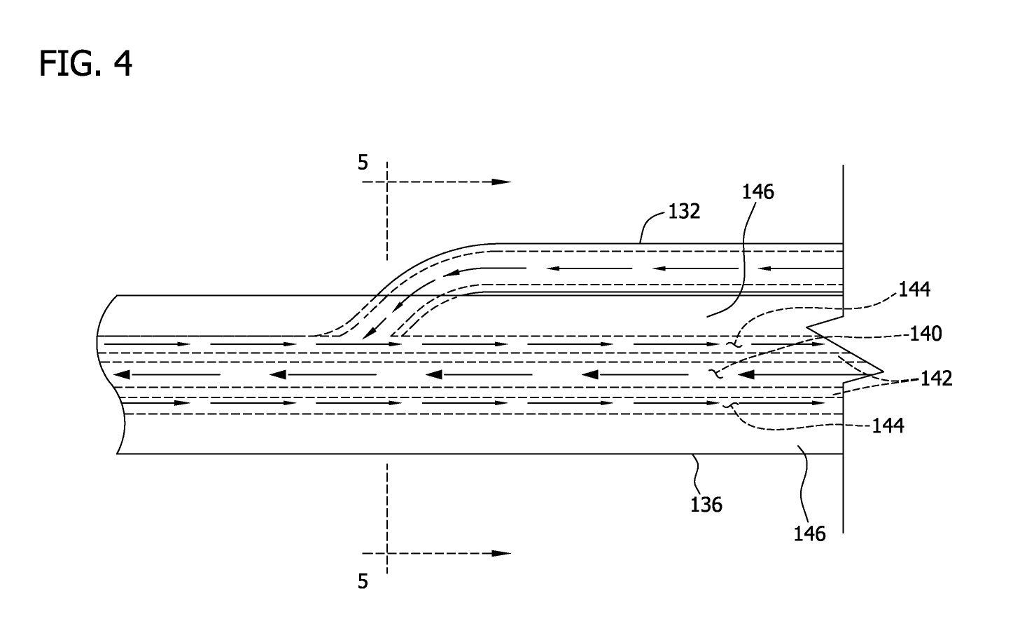

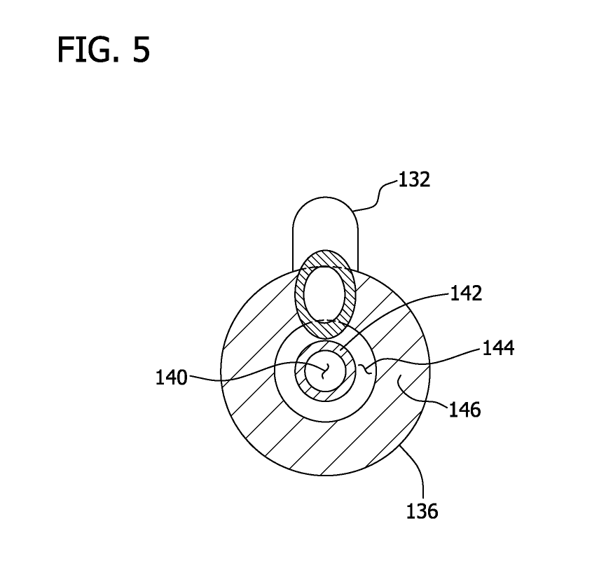

As shown in FIGS. 4 and 5, the second outlet 126 of the valve 120 is in fluid communication with the infusion line 110 of the infusion system 100. In this embodiment, the second outlet 126 is in fluid communication with the infusion line 110 adjacent the outlet end 114 of the infusion line, e.g., tubing 132 connects the second outlet 126 to the infusion line 110 adjacent the outlet end 114. The enlarged views of FIGS. 4 and 5 show the junction of the tubing 132 and the infusion line 110 in greater detail. The portion of the tubing 132 adjacent the outlet end 114 has a single outer lumen 144, although it may instead have a different number of outer lumens. In other embodiments, the second outlet 126 may be connected to the infusion line 110 at other locations on the infusion line (e.g., adjacent the inlet end 112 of the infusion line).

In a first position, the valve 120 directs gas from the source of gas 106 to the first outlet 124 and into the container of ophthalmic fluid 102. In a second position, the valve 120 directs gas from the source of gas 106 to the second outlet 126 and into the infusion line 110 of the infusion system 100. In yet a third position (i.e., a “closed” position), the valve 120 prevents gas from exiting either of the first outlet 124 and the second outlet 126. The valve 120 can be moved between the positions manually by a user turning a valve handle 134, by an electro-mechanical actuator connected to the valve and a control system, or by other suitable means.

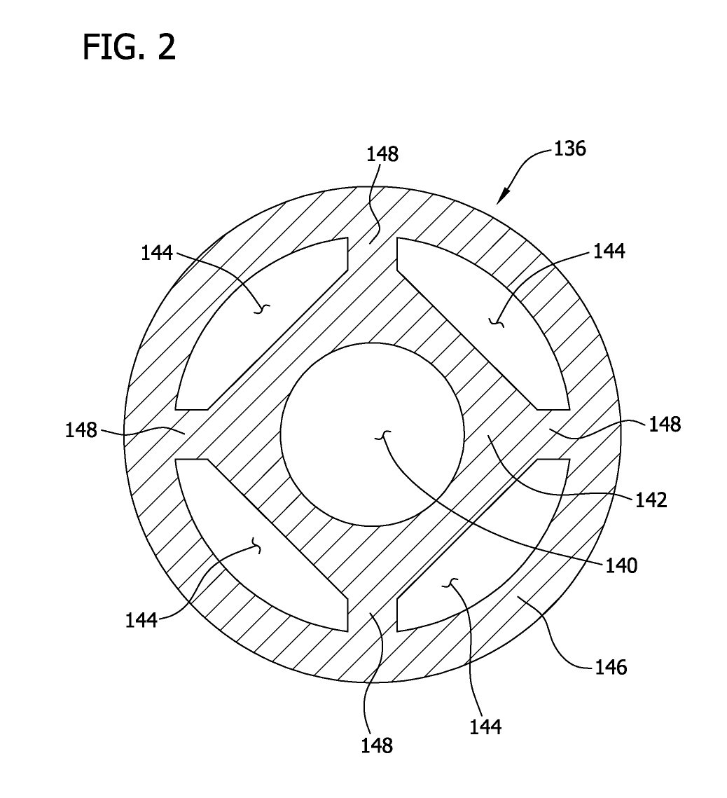

As shown in FIG. 1, a portion of the infusion line 110 is comprised of a multi-lumen tube 136 (referred to interchangeably as the “tube”). The tube generally extends from a vent connector 162 at one end to a junction of the tubing 132 and the infusion line 110 adjacent the outlet end 114. The tube 136 is shown in greater detail in the cross-sectional view of FIG. 2. The tube 136 has an inner lumen 140 having an inner shell 142 surrounded by four outer lumens 144, although other embodiments may have a different number of outer lumens. An outer shell 146 forms the outer surface of the tube 136. The outer lumens 144 are separated from the inner lumen 140 by webs 148. The inner shell 142 prevents gas/fluid disposed in the inner lumen 140 from mixing with gas/fluid disposed in the outer lumens 144 and vice versa.

Returning to FIG. 1, an outlet valve 150 is positioned near the outlet end 114 of the infusion line 110 to control the flow of gas/fluid through the inner lumen 140 of the infusion line.

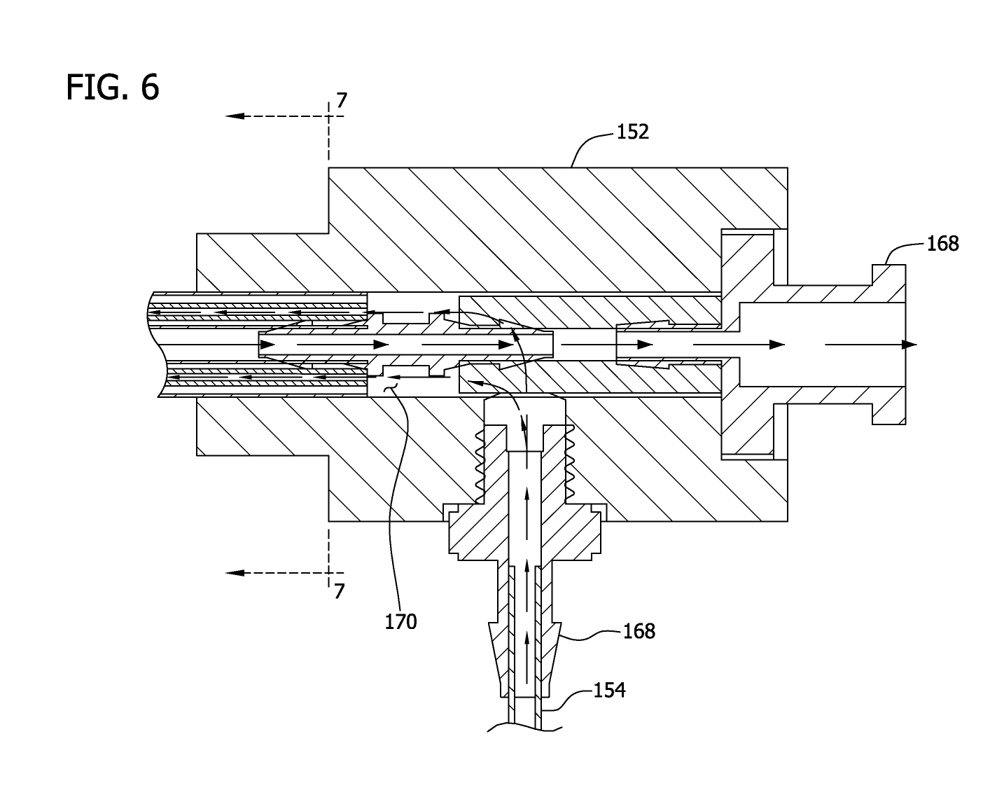

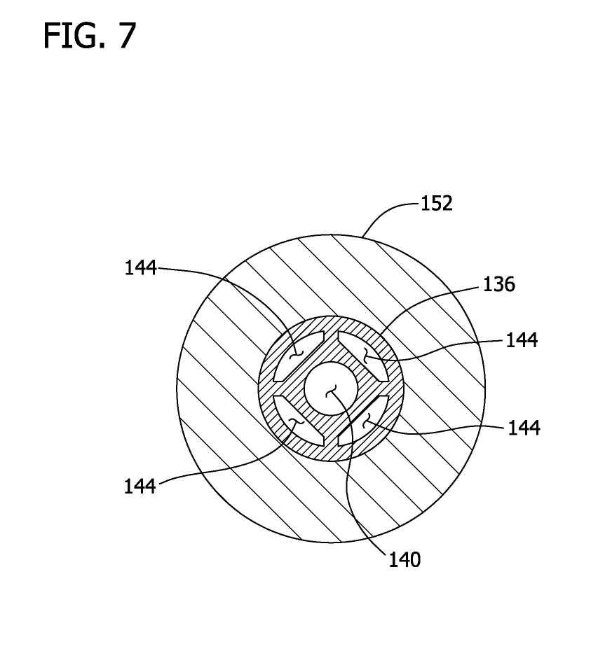

Bypass connectors 152 are positioned on opposing sides of the outlet valve 150 and are shown in greater detail in FIGS. 6 and 7. The bypass connectors 152 are connected by tubing 154 coupled to connectors 168 to connect the outer lumens 144 of the infusion line 110 on opposing sides of the outlet valve. The connectors 168 are in fluid communication with a void 170 in the bypass connectors 152 that is in turn in fluid communication with the outer lumens 144.

Gas flows through the tubing 154 into the connector 168, through the void 170 and into the outer lumens 144 in the bypass connector 152 shown in FIGS. 6 and 7. In the other bypass connector 152 (shown only in FIG. 1) gas flows in the opposite direction (i.e., the flow of gas enters the bypass connector through outer lumens 144 and leaves through the connector 168) during normal use, although gas can flow in either direction according to different embodiments.

The outlet valve 150 in this embodiment does not control the flow of gas through the outer lumens 144. However, in other embodiments, the outlet valve 150 may control the flow of gas through the outer lumens 144. Moreover, in still other embodiments another valve may be used to control the flow of gas in the outer lumens 144.

An inlet valve 160 is positioned near the inlet end 112 of the infusion line 110 to control the flow of gas/fluid through the inner lumen 140 of the infusion line. In other embodiments, the inlet valve 160 is not used. A vent connector 162 is positioned between the inlet valve 160 and the outlet valve 150, but nearer to the inlet valve. This vent connector 162 permits gas/fluid disposed in the outer lumens 144 to vent from the outer lumens. Tubing may be connected to the vent connector 162 in some embodiments to direct gas to a storage or processing system. A drip chamber 164 is positioned inline with the infusion line 110 between the hub 128 and the inlet valve 160. Luers 166 are used to connect various tubes to other components of the system 100.

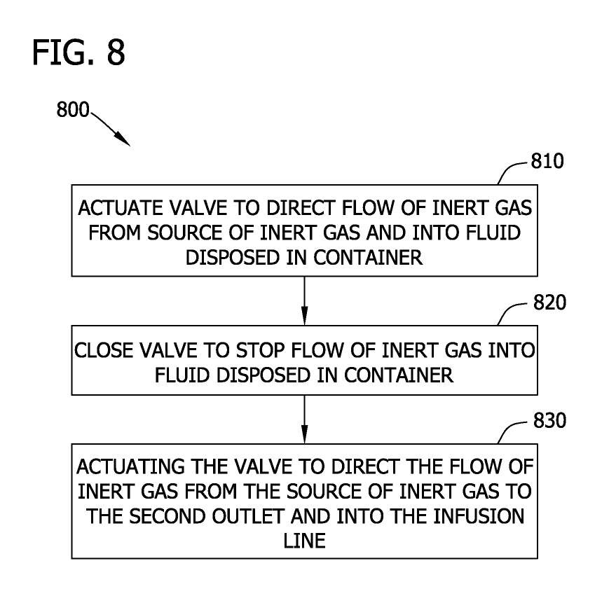

As shown in FIG. 8, a method 800 of deoxygenating ophthalmic fluid begins in block 810 with actuating or moving a valve, such as the valve 120, to the first position to direct a flow of gas from the source of gas 106. As described above, the flow will move through the first outlet 124 of the valve 120. The flow will then move through the hub 128 and into the ophthalmic fluid 104 disposed in the container 102. The valve 120 may be actuated manually by a user (e.g., a surgeon), by an electro-mechanical actuator connected to a control system, or by other suitable means.

As the gas flows through the hub 128 and into the ophthalmic fluid 104 disposed in the container 102, the gas deoxygenates the fluid by purging or displacing oxygen dissolved in the fluid. The purged or displaced oxygen and gas that has travelled through the ophthalmic fluid 104 (i.e., “spent” gas) then exits the container 102 through the hub 128. The purged oxygen and spent gas travel through the inner lumen 140 of the infusion line 110 before exiting the infusion line through the outlet end 114. This flow of oxygen and spent gas purges the inner lumen 140 of oxygen or gaseous contaminants that may be present in the inner lumen. In other embodiments, the flow of oxygen and spent gas may be directed through the outer lumens 144. In still other embodiments, the oxygen and spent gas may be vented to the atmosphere and do not pass through either the inner lumen 140 or the outer lumens 144.

In block 820, the valve 120 is closed to stop the flow of gas into the ophthalmic fluid 104 disposed in the container 102. Flow is stopped after a period of time sufficient for the ophthalmic fluid 104 to be deoxygenated below a predetermined level, as further described below. The amount of time required to deoxygenate the ophthalmic fluid 104 below this predetermined level may be determined based on experimental data or other tests previously conducted on ophthalmic fluid.

In other embodiments, the oxygen content of the ophthalmic fluid 104 may be monitored by a sensor or other similar device to determine when to close the valve 120. In these embodiments the valve 120 is closed when the sensor determines that the fluid 104 has been deoxygenated below the predetermined level.

In block 830, the valve 120 is actuated or moved to the second position to direct the flow of gas from the source of gas 106 to the second outlet 126 and into the outer lumens 144 of the infusion line 110. The valve 120 may be actuated to the second position before or after the inlet valve 160 and the outlet valve 150 is opened.

As described above, the second outlet 126 of the valve is connected by tubing 132 to the outer lumens 144 of infusion line 110 adjacent the outlet end 114 of the infusion line. The flow of gas travels into the outer lumens 144 and progresses towards the inlet end 112 of the infusion line 110. The gas then exits the outer lumens 144 through the vent connector 162.

The flow of gas through the outer lumens 144 may continue while ophthalmic fluid 104 flows through the inner lumen 140. The gas may act as a shield or buffer that prevents oxygenation of the ophthalmic fluid 104 as it flows through the inner lumen 140. The flow of gas through the outer lumens 144 purges the outer lumens of oxygen and/or other contaminants. This decreases or eliminates oxygen diffusion through the inner shell 142 and thereby decreases or eliminates oxygen dissolution in the fluid 104 within the inner lumen 140. To the extent that the tubing 132 decreases re-oxygenation of the fluid 104 as a result of this shielding effect, the tubing may have any configuration that generally permits shielding of the inner lumen 140 with a non-reactive gas.

In other embodiments, the second outlet 126 of the valve 120 may be connected to the inner lumen 140 of the infusion line 110 and gas may then be directed into the inner lumen. This flow of gas might act to purge the inner lumen 140 of any contaminants contained in the inner lumen prior to ophthalmic fluid 104 flowing through the inner lumen. Moreover, this flow of gas might instead act to enter the eye through the infusion cannula 116.

After the valve 120 is actuated to the second position, the inlet valve 160 and the outlet valve 150 are opened to permit the ophthalmic fluid 104 to flow from the container 102 and through the infusion line 110. The ophthalmic fluid 104 then exits the infusion line 110 through the infusion cannula 116 directly into the eye. The inlet valve 160 and the outlet valve 150 are closed after a desired volume of ophthalmic fluid 104 is discharged into the eye or remain open during a surgical procedure.

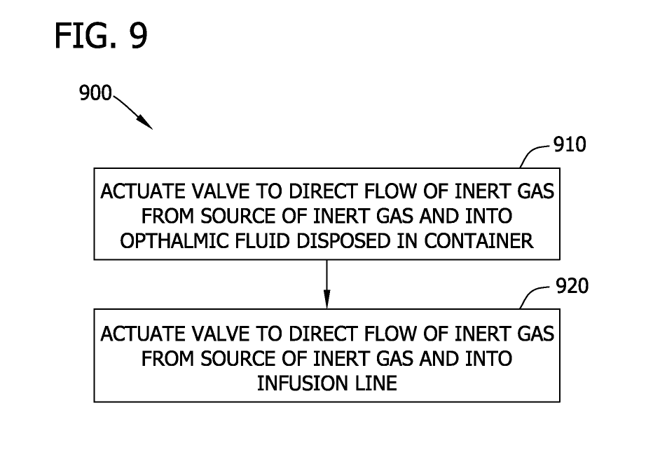

FIG. 9 depicts another method 900 for deoxygenating ophthalmic fluid 104 disposed in the container 102. In this embodiment, the ophthalmic fluid 104 is deoxygenated such that the oxygen contained in the fluid has a partial pressure of less than 10 mmHg. The method begins in block 910 with actuating or opening a valve, such as the valve 120, to direct the flow of gas from the source of gas 106 to the first outlet 124 and thereby direct it into the ophthalmic fluid 104 disposed in the container 102.

As the gas flows through the hub 128 and into the ophthalmic fluid 104 disposed in the container 102, the gas deoxygenates the fluid 104 by purging or displacing oxygen dissolved in the fluid, as described above in relation to FIG. 8.

In block 920, the valve 120 is actuated or moved to its second position such that gas is directed from the source of gas 106 to the second outlet 126 and into the infusion line 110 when the ophthalmic fluid 104 has been deoxygenated below a predetermined level.

The amount of time required to deoxygenate the ophthalmic fluid 104 below this predetermined level may be determined based on experimental data or other tests previously conducted on ophthalmic fluid. After the valve 120 has been open for the required amount of time, the ophthalmic fluid has been deoxygenated such that the oxygen content of the ophthalmic fluid 104 has been reduced below the predetermined level. The valve 120 is then actuated to its second position. In other embodiments, the deoxygenation of the ophthalmic fluid 104 may be monitored by a sensor or other similar device while the fluid is deoxygenated. In these embodiments the valve 120 may be actuated to its second position when the sensor indicates that the ophthalmic fluid 104 has been deoxygenated below the predetermined level.

After the valve 120 is actuated to the second position, the inlet valve 160 and the outlet valve 150 are opened to permit the ophthalmic fluid 104 to flow from the container 102 and through the infusion line 110. The ophthalmic fluid 104 then exits the infusion line 110 through the infusion cannula 116 and enters the eye. The inlet valve 160 and the outlet valve 150 are closed after a desired volume of ophthalmic fluid 104 is discharged into the eye or remain open during a surgical procedure.

Prior research has indicated that oxygenated ophthalmic fluid can cause oxidative stress within the eye that damages various proteins. This damage can result in the opacification of the crystalline lens of the eye.

During use, the infusion system 100 is operable to reduce the oxygen content of ophthalmic fluid 104 disposed in the container 102 and injected into the eye. In the exemplary embodiment, the ophthalmic fluid 104 is deoxygenated such that the oxygen remaining in the fluid has a partial pressure of less than or equal 10 mmHg. Deoxygenation of the fluid 104 to such a level results in the fluid having an oxygen content that is similar to that of the naturally occurring vitreous within the eye. The injection of this deoxygenated fluid reduces the likelihood of complications following a vitrectomy.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. The use of terms indicating a particular orientation (e.g., “top”, “bottom”, “side”, etc.) is for convenience of description and does not require any particular orientation of the item described.

As various changes could be made in the above constructions and methods without departing from the scope of the invention, it is intended that all matter contained in the above description and shown in the accompanying drawing[s] shall be interpreted as illustrative and not in a limiting sense.

What is claimed is:

1. A method of injecting deoxygenated ophthalmic fluid into an eye, the method comprising:
actuating a valve of an infusion system to direct a flow of gas from a source of gas into a container of oxygenated ophthalmic fluid, wherein the infusion system includes the valve in fluid communication with the source of gas, a first outlet in fluid communication with the container of ophthalmic fluid, and a second outlet in fluid communication with an infusion line of the system;
closing the valve to stop the flow of gas into the fluid disposed in the container when the fluid has been deoxygenated below a predetermined level; and
actuating the valve to direct the flow of gas from the source of gas to the second outlet and thereby into the infusion line.

2. The method of claim 1 further comprising opening an inlet valve of the infusion line to direct deoxygenated ophthalmic fluid from the container through the infusion line into the eye.

3. The method of claim 1 wherein the flow of gas is directed from the second outlet and into the infusion line adjacent an outlet end of the infusion line.

4. The method of claim 1 wherein the infusion line comprises a multi-lumen tube having an inner lumen and an outer lumen.

5. The method of claim 4 wherein the flow of gas is directed from the second outlet and into the inner lumen of the infusion line to purge the inner lumen.

6. The method of claim 4 wherein the flow of gas is directed from the second outlet and into the outer lumen of the infusion line.

7. The method of claim 6 wherein the flow of gas is directed from the second outlet and into the outer lumen of the infusion line adjacent an outlet end of the infusion line.

8. The method of claim 1 wherein the ophthalmic fluid is deoxygenated such that a partial pressure of oxygen remaining in the fluid is less than 10 mmHg.

9. A method of delivering deoxygenated ophthalmic fluid into an eye, the method comprising:
actuating a valve of an infusion system to direct a flow of gas from the source of gas into a container of oxygenated ophthalmic fluid, wherein the infusion system includes the valve in fluid communication with the source of gas, a first outlet in fluid communication with the source of gas, and a second outlet in fluid communication with an infusion line of the system, wherein the flow of gas deoxygenates the ophthalmic fluid;
actuating the valve to direct the flow of gas from the source of gas and into the infusion line when the fluid has been deoxygenated below a predetermined level; and
opening an outlet valve of the infusion line to deliver a flow of deoxygenated ophthalmic fluid from the container through the infusion line into the eye.

10. The method of claim 9 wherein the flow of gas is directed from the source of gas and into the infusion line adjacent an outlet end of the infusion line where ophthalmic fluid is discharged from the infusion line into an eye.

11. The method of claim 10 wherein the flow of gas is directed into an outer lumen of the infusion line adjacent the outlet end of the infusion line.

12. The method of claim 9 wherein the infusion line comprises a multi-lumen tube having an inner lumen and an outer lumen.

13. The method of claim 12 wherein the flow of gas is directed from the second outlet and into the inner lumen of the infusion line to purge the inner lumen prior to use of the infusion line to convey ophthalmic fluid into an eye.

14. The method of claim 12 wherein the flow of gas is directed from the second outlet and into the outer lumen of the infusion line.

15. The method of claim 9 wherein the ophthalmic fluid is deoxygenated such that a partial pressure of oxygen remaining in the fluid is less than 10 mmHg.

16. A system for deoxygenating an ophthalmic fluid disposed in a container, the system comprising:
an ophthalmic infusion apparatus including a multi-lumen infusion line having a fluid outlet for discharging ophthalmic fluid into an eye, the infusion line having an inner lumen and an outer lumen;
a valve having an inlet in fluid communication with a source of gas, a first outlet, and a second outlet, the valve operable to selectively direct gas to one of the first outlet and the second outlet;
wherein the first outlet of the valve is in fluid communication with the container of ophthalmic fluid; and
wherein the second outlet of the valve is in fluid communication with the infusion line adjacent the fluid outlet.

17. The system of claim 16 wherein the ophthalmic infusion apparatus is configured such that gas is directed into the infusion line.

18. The system of claim 17 wherein the gas is directed into the inner lumen of the infusion line.

19. The system of claim 17 wherein gas exits the ophthalmic fluid infusion apparatus through the fluid outlet of the apparatus.

20. The system of claim 16 wherein the second outlet of the valve is in fluid communication with the inner lumen of the infusion line.

21. The system of claim 16 wherein the second outlet of the valve is in fluid communication with the outer lumen of the infusion line.

22. The system of claim 16 further comprising a source of gas in fluid communication with the inlet of the valve.

23. An ophthalmic fluid infusion apparatus for deoxygenating ophthalmic fluid injected into an eye, the apparatus comprising:
a multi-lumen infusion line having a fluid outlet for discharging fluid into the eye, the infusion line having an inner lumen and an outer lumen;
a valve having an inlet in fluid communication with a source of gas, a first outlet, and a second outlet, the valve operable to selectively direct gas from the inlet to one of the first outlet and the second outlet;
a container of ophthalmic fluid;
wherein the first outlet of the valve is in fluid communication with the container of ophthalmic fluid, wherein gas directed from the first outlet into the fluid results in the deoxygenation of the fluid; and
wherein the second outlet of the valve is in fluid communication with the ophthalmic infusion apparatus adjacent the fluid outlet of the infusion line.

24. The apparatus of claim 23 wherein the second outlet of the valve is in fluid communication with the outer lumen of the infusion line adjacent the fluid outlet of the line.

25. The system of claim 23 further comprising a source of gas in fluid communication with the inlet of the valve, the gas being nitrogen.

26. The apparatus of claim 23 further comprising a hub for connecting the first outlet of the valve to the container of ophthalmic fluid and the infusion line such that the first outlet is in fluid communication with the container.

27. The apparatus of claim 26 wherein the hub is configured to direct oxygen and gas that has passed through the fluid in the container into the inner lumen of the infusion line.

28. The apparatus of claim 26 wherein the hub is configured to direct oxygen and gas that has passed through the fluid in the container into the outer lumen of the infusion line.
